(19) United States
(12) Patent Application Publication
(10) Pub. No.: US 2026/0008734 A1
(43) Pub. Date: Jan. 8, 2026

(54) LOW-TEMPERATURE DEPOLYMERIZATION OF POLYMER CONTAINING URETHANE FUNCTIONAL GROUP USING COSOLVENT AND METHOD FOR PRODUCING POLYOL

(71) Applicant: KOREA RESEARCH INSTITUTE OF CHEMICAL TECHNOLOGY, Daejeon (KR)

(72) Inventors: Joung Mo CHO, Daejeon (KR); Thi Hong Ngan LE, Daejeon (KR); Ye Rim JO, Daejeon (KR)

(73) Assignee: KOREA RESEARCH INSTITUTE OF CHEMICAL TECHNOLOGY, Daejeon (KR)

(21) Appl. No.: 19/327,694

(22) Filed: Sep. 12, 2025

Related U.S. Application Data
(63) Continuation of application No. PCT/KR2024/000519, filed on Jan. 11, 2024.

(30) Foreign Application Priority Data
Jan. 13, 2023 (KR) 10-2023-0005562

(51) Int. Cl. C07C 29/128; C08J 11/24

(52) U.S. Cl. CPC C07C 29/1285; C08J 11/24

(57) ABSTRACT

The present disclosure relates to a method for low-temperature depolymerization of a polymer containing a urethane-functional group using a cosolvent and a method for producing polyol. More specifically, the present disclosure relates to a method of adding a compound having two or more alcohol-functional groups as a depolymerization solvent for decomposing a polymer containing a urethane-functional group, adding an aromatic compound having an alkoxy-functional group as a cosolvent to construct a reaction system for decomposing the polymer containing the urethane-functional group, thereby performing rapid depolymerization at a low temperature, and obtaining high-quality recycled polyol in high yield through a physical separation process of the reaction product generated therefrom.

### CROSS-REFERENCE TO RELATED APPLICATIONS

This application is a continuation of PCT International Patent Application No. PCT/KR2024/000519 which has an International filing date of Jan. 11, 2024, and which claims priority to Korean Patent Application No. 10-2023-0005562 filed Jan. 13, 2023, the entire contents of each of which are incorporated herein by reference.

### TECHNICAL FIELD

The present disclosure relates to a composition for low-temperature depolymerization of urethane-functionalized polymers such as waste polyurethane, a depolymerization method, and a method for manufacturing polyol therefrom. More specifically, the disclosure relates to a method for adding a compound with two or more alcohol functional groups as a depolymerization solvent to decompose a urethane-functionalized polymer, adding a cosolvent with one or more alkoxy functional groups to form a reaction system for decomposing the urethane-functionalized polymer, performing rapid depolymerization at a low temperature, and obtaining a high-quality recycled polyol at a high yield through a physical separation process of the resulting reaction products.

### BACKGROUND ART

Polyurethane is a polymer compound in which the main polymer chain is structured by urethane bonds. It is synthesized through a urethane polymerization reaction between a polyol with alcohol functional groups and an isocyanate compound with isocyanate functional groups at both ends. Polyurethane forms a polymer structure through a soft segment (SS), derived from the polyol, which provides a flexible structure and elastic recovery, and a hard segment (HS), which provides strong cohesive force through isocyanate and urethane bonds. Because the types of polyols and isocyanates used in synthesis are diverse and their physical properties can be controlled according to the blending conditions of the raw materials, they are applied in a wide range of materials.

Based on its physical properties, polyurethane can be classified into flexible foam, rigid foam, and semi-rigid foam. It is also categorized and utilized as foamed polyurethane (Polyurethane foam; PUF), which is manufactured into a lightweight, honeycomb-like structure by intense bubble generation during the manufacturing or polymerization process, and non-foamed polyurethane, which is mainly applied in coatings (C), adhesives (A), sealants (S), and elastomers (E).

Polyurethane is a versatile plastic product widely used in a variety of applications due to its excellent physical properties and processability. These include soft cushion materials like clothing, bed mattresses, and car seats, as well as thermal insulators, soundproofing materials, flooring materials, and various other construction materials used in refrigerators, freezers, and insulation pipes.

As materials with various properties have been developed, urethane is being applied in various ways in modern daily life, and it is known that not only the scale of related industries but also the amount of waste has greatly increased. In particular, compared to other materials, the utilization cycle is short, and the final form of the material is often manufactured by cutting after synthesis rather than being directly molded through polymerization, so it is one of the plastic products with a very large amount of process waste.

In particular, many polyurethane wastes have thermosetting properties that harden when heat is applied, making it difficult to melt and recycle them, and since there is no appropriate treatment method, most of them are incinerated, and in this process, secondary pollutants such as harmful gases and dust are generated, causing many environmental problems. Recently, as the seriousness of environmental problems caused by synthetic polymer wastes has emerged, research on recycling technologies that can reduce the amount of waste plastics discharged and minimize adverse effects on the environment is being actively conducted, but recycling technologies for polyurethane have been relatively few due to economic feasibility, quality of final products, and technical limitations.

Technologies for recycling polyurethane include: physical recycling, which involves crushing the material without changing its chemical structure and processing it into a different form; thermal recycling, which involves heating it to convert it into fuel or directly recovering thermal energy; and chemical recycling, which involves recovering some or all of the material back to its pre-synthesis raw material form through a chemical reaction.

Thermal recycling methods can recover only energy by heating or produce low-grade oil by completely deforming the polymer structure through pyrolysis. However, these methods are environmentally unfriendly since the material is consumed as fuel and cannot be repeatedly regenerated.

Physical recycling methods are mostly used to produce lower-grade or lower-quality materials and have very limited uses and applications, and the number of recycling cycles is restricted, so they cannot be a perfect recycling method to overcome environmental problems.

In contrast, the chemical recycling method can decompose polyurethane and return it to its pre-synthesis raw materials. This is an environmentally friendly recycling method that enables the infinite, repetitive use of resources, as it can substitute a portion of polyurethane, which was previously synthesized using only petroleum-derived raw materials, with recycled raw materials.

The method of chemically recycling polyurethane proceeds through a reaction in which a solvent and a catalyst are contacted with waste polymer and heat is applied to decompose the urethane bond, and is classified into hydrolysis, glycolysis, acidolysis, aminolysis, etc., depending on the type of solvent added. Among these chemical decomposition (or depolymerization) reaction paths, the glycolysis method, which proceeds well under relatively mild conditions and is easy to purify, is the most commonly applied depolymerization method.

As a prior art, U.S. Pat. No. 4,243,560 (published Jan. 6, 1981) discloses a technology for obtaining recyclable alcohols and polyols as distilled products by heating and decomposing flexible or semi-rigid polyurethane foam at a high temperature without oxygen exposure. This technology is a pyrolysis technique that does not use any additives or catalysts, and it has the characteristic of being able to induce decomposition into products in a short residence time of a few minutes to an hour after the raw material is introduced. However, since the depolymerization is performed at a high temperature of 450° C. to 600° C., energy consumption can be excessive, and a high investment cost is required for the high-temperature reaction, which are disadvantages. To overcome these problems, chemical depolymerization technologies have been developed that add a polyhydric alcohol solvent as a reactant to promote polymer decomposition by substituting alcohol functional groups after breaking urethane bonds, and add a catalyst to allow the depolymerization reaction to proceed at a temperature below the boiling point of the solvent.

For example, U.S. Pat. No. 6,750,260 (published Jun. 15, 2004) discloses a method for performing glycolysis and depolymerization at a temperature of 180° C. to 200° C. using a polyhydric alcohol solvent such as diethylene glycol as a reactant and KOH as a catalyst. It also discloses a method for manufacturing a recycled polyol by further adding a compound with an epoxy group, such as propylene oxide or ethylene oxide, to the polyol intermediate obtained as a depolymerization product. In the glycolysis reaction, which uses a polyhydric alcohol as a solvent for the reaction to induce polyurethane decomposition, polyol and diamine compounds are obtained as reaction products when urethane bonds are broken. However, some of the products are obtained as a dark, opaque, and turbid black mixture due to side reactions from contact with oxygen or compounds containing oxygen. The depolymerization products obtained this way have limitations in their re-synthesis or re-utilization as a polyurethane material of the same or similar quality, even if they contain a large number of active alcohol groups.

U.S. Pat. No. 6,020,386 (published Feb. 1, 2000) discloses a glycolysis depolymerization method that minimizes the generation of diamine compounds in the depolymerization product by adding a dialkyl carbonate or a dicarbonyl compound.

Korean Registered Patent No. 10-0278099 (published Jan. 15, 2001) discloses a method for depolymerizing a polyurethane elastomer through a glycolysis reaction, then adding a compound with a polyhydric carboxylic acid functional group to lower the residual concentration of the glycol used as a solvent in the produced reactant. This induces an ester reaction of the glycol to convert it into a high molecular weight compound, after which the resulting product is used as a raw material for recycled polyurethane. This technology proposes reaction conditions for performing glycolysis at a reaction temperature of 120° C. to 300° C. for 30 minutes to 15 hours. However, at low temperatures, the depolymerization rate is very slow, resulting in a very low product yield. At high temperatures, the depolymerization rate can be increased, but side reactions can be promoted, making it difficult to produce high-quality recycled raw materials.

Meanwhile, depolymerization products contain compounds such as diamines in addition to polyols. However, because the depolymerization product itself is applied to the re-synthesis of polyurethane without separating these compounds, it can be difficult to control the quality of the manufactured recycled polyurethane. Korean Registered Patent No. 10-1447247 (published Oct. 8, 2014) discloses a method for manufacturing a recycled polyol by mixing and heating raw materials with a polyhydric alcohol, then applying an amine-based oligomer, animal/vegetable oil, and a Bi, Mo-based catalyst to a reaction at a reaction temperature of 170° C. to 200° C., and then applying an anti-emulsifying agent to separate the recycled polyol to improve the color of the recycled polyol and the recycled polyurethane produced from it.

Conventional technologies for chemically decomposing polyurethane by adding polyhydric alcohols as solvents for recycling are performed under high-temperature reaction conditions close to 200° C. To improve the quality (color, reactivity, etc.) of the recovered recycled polyol, a complicated purification process is required. This includes continuously adding nitrogen during the depolymerization process to minimize contact with outside air or configuring reaction conditions that can suppress side reactions caused by oxide inflow. Therefore, a high operating cost is required to manufacture products of consistent quality, and there is still much room for improvement in terms of energy efficiency.

The present disclosure aims to overcome the disadvantages of the conventional art by applying a hydrophobic cosolvent with a strong interaction with the hard segment (HS) adjacent to the urethane bond as a component of the reaction. This lowers the activation energy of the depolymerization reaction, thereby promoting rapid depolymerization even at a relatively low reaction temperature. It also aims to provide an environment that can minimize side reactions due to oxidation during the decomposition of polyurethane.

Furthermore, the present disclosure aims to propose an efficient and new glycolysis reaction pathway from the depolymerization of urethane-functionalized polymers such as waste polyurethane. This pathway makes it easy to recover the polyol generated by the depolymerization of urethane, and the resulting polyol does not have a dark color, making it a high-quality polyol raw material that can be used in the production of various polyurethanes.

### DISCLOSURE OF THE INVENTION

### Technical Problem

The present disclosure aims to provide an effective and economical composition for the depolymerization of urethane-functionalized polymers, a method for depolymerizing said polymers, and a method for manufacturing high-quality polyol at a high yield therefrom. These methods improve the reaction performance in the depolymerization of urethane-functionalized polymers according to the glycolysis reaction pathway.

### Technical Solution

To solve the above-mentioned problems, the present disclosure provides a depolymerization composition for urethane-functionalized polymers, characterized by comprising (1) a compound with two or more alcohol functional groups (—OH); and (2) an aromatic compound with one or more alkoxy functional groups.

The compound with two or more alcohol functional groups may be one or more selected from the group consisting of ethylene glycol, trimethylene glycol, 1,2-propanediol, tetramethylene glycol, neopentyl glycol, pentamethylene glycol, hexamethylene glycol, decamethylene glycol, dodecamethylene glycol, 1,4-cyclohexanedimethanol, diethylene glycol, triethylene glycol, tetraethylene glycol, polyethylene glycol, dipropylene glycol, tripropylene glycol, tetrapropylene glycol, polypropylene glycol, di(tetramethylene) glycol, tri(tetramethylene) glycol, polytetramethylene glycol, pentaerythritol, 2,2-bis(4-β-hydroxyethoxyphenyl) propane, glycerol, pentanetriol, and hexanetriol. The aromatic compound with one or more alkoxy functional groups may be one or more selected from the group consisting of methoxybenzene, 1,2-dimethoxybenzene, 1,3-dimethoxybenzene, 1,4-dimethoxybenzene, 1,2,3-trimethoxybenzene, 1,2,4-trimethoxybenzene, 1,3,5-trimethoxybenzene, 1,2,3,4-tetramethoxybenzene, 1,2,3,5-tetramethoxybenzene, 1,2,4,5-tetramethoxybenzene, 1-methoxy-2-methylbenzene, 1-methoxy-3-methylbenzene, 1-methoxy-4-methylbenzene, ethoxybenzene, and butoxybenzene.

In one embodiment of the present disclosure, the compound with two or more alcohol functional groups and the aromatic compound with one or more alkoxy functional groups may be mixed in a weight ratio of 1:20 to 20:1.

The depolymerization composition of the present disclosure may further comprise one or more depolymerization catalysts for urethane-functionalized polymers selected from the group consisting of metal catalysts such as alkali hydroxides, alkaline-earth hydroxides, alkali acetates, alkaline-earth acetates, alkali carbonates, alkali bicarbonates, alkaline-earth carbonates, and alkali oxides, or guanidine-based or amine-based organic compounds.

In addition, the present disclosure provides a method for depolymerizing a urethane-functionalized polymer, characterized by comprising a step of contacting a mixed solvent containing a compound with two or more alcohol functional groups and an aromatic compound with one or more alkoxy functional groups with the urethane-functionalized polymer.

In one embodiment of the depolymerization method of the present disclosure, the mass of the urethane-functionalized polymer may be in the range of 1 wt % to 200 wt % based on the mass of the mixed solvent containing the compound with two or more alcohol functional groups and the aromatic compound with one or more alkoxy functional groups.

In another embodiment of the depolymerization method of the present disclosure, the mixed solvent may have a temperature in the range of 100° C. to 170° C. The contact between the mixed solvent and the urethane-functionalized polymer may be carried out in the presence of one or more catalysts selected from the group consisting of metal catalysts such as alkali hydroxides, alkaline-earth hydroxides, alkali acetates, alkaline-earth acetates, alkali carbonates, alkali bicarbonates, alkaline-earth carbonates, and alkali oxides, or guanidine-based or amine-based organic compounds. The total mass of the catalyst may be in the range of 0.0001 times to 0.5 times the mass of the urethane-functionalized polymer.

In another embodiment of the depolymerization method of the present disclosure, a step of purging with an inert gas is additionally included before or at the beginning of the contact between the mixed solvent and the urethane-functionalized polymer.

In yet another embodiment of the depolymerization method of the present disclosure, the method may be characterized by inducing phase separation by cooling the resulting liquid reaction mixture after the step of contacting the mixed solvent with the urethane-functionalized polymer.

In addition, the present disclosure provides a method for manufacturing polyol by depolymerizing a urethane-functionalized polymer, characterized by comprising a step of contacting a mixed solvent containing a compound with two or more alcohol functional groups and an aromatic compound with one or more alkoxy functional groups with the urethane-functionalized polymer to decompose the urethane bonds within the polymer and obtain polyol.

### Advantageous Effects

In the present disclosure, it is possible to depolymerize a polymer containing a urethane functional group without using high-temperature, high-pressure reaction conditions and to depolymerize a polymer containing a urethane functional group capable of producing high-quality polyol therefrom.

According to the present disclosure, by applying a mixed solvent, which is a mixture of a compound with two or more alcohol functional groups (—OH) and a cosolvent, which is an aromatic compound with one or more alkoxy functional groups, to the depolymerization of a urethane-functionalized polymer, the low-density solid urethane-functionalized polymer, such as polyurethane foam, can dissolve better in the reaction solvent. This allows it to be prepared as a liquid depolymerization reactant in a short time (or short cycle), and during the depolymerization step, it significantly enhances the depolymerization reaction rate by lowering the reaction activation energy barrier, unlike conventional reactions.

In addition, the aromatic compound with one or more alkoxy functional groups forms a strong intermolecular attraction (π-π and hydrogen bonding) with the part forming the urethane bond within the urethane-functionalized polymer. This may more dominantly induce a nucleophilic attack by the depolymerization catalyst or polyhydric alcohol. Because it is hydrophobic, it may also significantly suppress side reactions, enabling the complete decomposition of polyurethane with a short reaction time at a low temperature below the boiling point of the constituent solvent during depolymerization. Since side reactions are suppressed, a high-quality polyol with almost no discoloration can be obtained as a depolymerization product.

The aromatic compound with one or more alkoxy functional groups used in the present disclosure has very limited solubility with the compound with two or more alcohol functional groups used as a reactant at temperatures below 100° C., but it mixes well with the polyol produced by depolymerization. Therefore, when the temperature of the reaction product is lowered to below 100° C. after the depolymerization reaction, most of the unreacted compound with two or more alcohol functional groups is concentrated in the hydrophilic solution layer and does not mix with the organic phase, which consists of a mixture of the aromatic compound with one or more alkoxy functional groups and the polyol product, thereby existing as a separate phase. From this, the unreacted compound with two or more alcohol functional groups can be separated from the reaction product easily and simply by a physical liquid-liquid extraction method. In addition, a high-purity polyol may be obtained as a final product by removing the cosolvent from the separated organic phase product using relatively simple methods such as evaporation, distillation, and extraction. Therefore, the present disclosure has the advantage of being able to economically obtain a transparent recycled polyol product that was difficult to obtain with conventional recycling methods. This polyol may then be used as a polymerization raw material to re-synthesize high-quality urethane-functionalized polymers.

The aromatic compound with one or more alkoxy functional groups used in the present disclosure may be naturally or artificially synthesized on a large scale, and most of them are biodegradable in a short period in nature, which may provide environmentally friendly operating conditions. A polyol raw material that may reproduce a material of the same or similar quality may be directly manufactured through the depolymerization of waste plastics. Therefore, the present disclosure may provide an environmentally friendly and economical method for the depolymerization of urethane-functionalized polymers and the manufacture of recycled polyol therefrom, and may provide a production process design method for this.

The depolymerization of urethane-functionalized polymers according to the present disclosure may be applied to the design of a simple process. Since depolymerization may proceed at a relatively low temperature and a high yield/high purity polyol may be obtained, it may provide a very effective and efficient chemical recycling method for waste plastics.

In addition, by returning the previously synthesized product to its pre-synthesis raw material, it may theoretically enable the infinite, repetitive use of the material, contributing to the realization of a circular plastics economy. Since the depolymerization reaction, product purification, and re-polymerization may be configured continuously together, it is possible to build an integrated chemical process that may produce recycled polymers on a large scale without the need for separate re-investment or equipment changes to existing polymer synthesis facilities.

### BRIEF DESCRIPTION OF THE DRAWINGS

FIG. 1 is a comparative graph illustrating the FT-IR spectra of the recycled polyol manufactured according to Example 3 of the present disclosure, the waste polyurethane of Raw Material 1 used as the depolymerization raw material, and the initial polyol raw material (virgin polyol) of Raw Material 2 used for its synthesis;

FIG. 2 is a view illustrating the state of the recycled polyol products manufactured according to Examples 1 to 10 and Comparative Examples 2 and 3 of the present disclosure;

FIG. 3 is a view illustrating the cross-sectional shape of the polyurethane foams manufactured using the recycled polyols manufactured according to Examples 1 to 10 and Comparative Examples 1 to 3 of the present disclosure, respectively;

FIG. 4 is a view illustrating the internal foam layer of the polyurethane foam manufactured from the recycled polyol according to some examples and comparative examples of the present disclosure, using a stereo microscope;

FIG. 5 is a view illustrating the state of the recycled polyol products manufactured by applying different types of cosolvents to the depolymerization reaction according to Examples 5, 11, and 12 of the present disclosure; and

FIG. 6 is a view illustrating the cross-sectional shape of the polyurethane foams manufactured using the recycled polyols manufactured according to Examples 5, 11, and 12 of the present disclosure, respectively.

### BEST MODE FOR CARRYING OUT THE INVENTION

Unless otherwise defined, all technical and scientific terms used in this specification have the same meaning as commonly understood by one of ordinary skill in the art to which this disclosure belongs. In general, the nomenclature used in this specification is well known and commonly used in the art.

Throughout this specification, when a part is said to “comprise” a certain component, it means that it may further comprise other components unless otherwise specified.

The depolymerization raw material of the present disclosure, a urethane-functionalized polymer such as polyurethane, may have a urethane functional group as the main bond connecting the constituent units of the polymer, for example, flexible or rigid polyurethane. It may include polyethylene, high-density polyethylene, low-density polyethylene, polypropylene, polyethylene terephthalate, or a combination thereof, but it is not limited to the aforementioned types of polymers. It may also be a mixture or copolymer with other known polymers, or it may contain various types of organic or inorganic foreign substances.

One example of the urethane-functionalized polymer is polyurethane, which is a multiblock copolymer containing urethane functional groups. It consists of a rigid hard segment (HS), which provides strong cohesive force and thermodynamic stability adjacent to the urethane bond, and a flexible soft segment (SS), which forms a flexible polymer chain and provides elasticity. It may be a polymer with various types, forms, and properties.

The part constituting the HS of the polyurethane may be various forms of diisocyanate, but aromatic diisocyanate is the most common. Representative examples of aromatic diisocyanates that form HS through the synthesis of polyurethane include monomeric forms such as toluene diisocyanate (TDI), methylene diphenyl diisocyanate (MDI), and m-xylene diisocyanate (MXDI). However, a polymeric form that is easy to handle because it dissolves easily in the organic phase, such as polyisocyanate, may also be used. The SS that constitutes the flexible polymer chain of the polyurethane may be in the form of polyether polyol or polyester polyol.

The present disclosure provides a composition for depolymerization of urethane-functionalized polymers, a method for depolymerizing urethane-functionalized polymers using said composition, and a method for manufacturing polyol through said depolymerization. The disclosure is characterized by using a compound with two or more alcohol functional groups as a reactant, adding an aromatic compound with one or more alkoxy functional groups as a cosolvent, and contacting the reaction mixture with the cosolvent with the urethane-functionalized polymer to decompose the urethane bonds within the polymer.

The depolymerization composition for urethane-functionalized polymers is characterized by comprising: (1) a compound with two or more alcohol functional groups (—OH); and (2) an aromatic compound with one or more alkoxy functional groups.

The compound with two or more alcohol functional groups may be a glycol with two alcohol functional groups in one molecule, glycerol with three alcohol functional groups, or a compound with four or more alcohol functional groups. Examples include dihydric alcohols such as ethylene glycol, trimethylene glycol, 1,2-propanediol, tetramethylene glycol, neopentyl glycol, pentamethylene glycol, hexamethylene glycol, decamethylene glycol, dodecamethylene glycol, 1,4-cyclohexanedimethanol, diethylene glycol, triethylene glycol, tetraethylene glycol, polyethylene glycol, dipropylene glycol, tripropylene glycol, tetrapropylene glycol, polypropylene glycol, di(tetramethylene) glycol, tri(tetramethylene) glycol, polytetramethylene glycol, pentaerythritol, and 2,2-bis(4-β-hydroxyethoxyphenyl) propane; trihydric alcohols such as glycerol, pentanetriol, and hexanetriol; polyhydric alcohols containing four or more alcohol functional groups; or a combination thereof.

The compound with two or more alcohol functional groups is a hydrophilic solvent. Since the catalysts added for depolymerization have characteristics close to hydrophilicity, the compound with two or more alcohol functional groups also functions as a solvent that dissolves the urethane-functionalized polymer as well as the catalyst in the reactant.

The aromatic compound with one or more alkoxy functional groups is an organic compound with at least one aromatic ring, where at least one of the hydrogens bonded to the carbons constituting the aromatic ring is substituted with an alkoxy functional group. It may be a liquid solvent that is hydrophobic at the depolymerization reaction temperature.

Examples of the aromatic compound with an alkoxy functional group include one or more compounds selected from the group consisting of methoxybenzene, 1,2-dimethoxybenzene, 1,3-dimethoxybenzene, 1,4-dimethoxybenzene, 1,2,3-trimethoxybenzene, 1,2,4-trimethoxybenzene, 1,3,5-trimethoxybenzene, 1,2,3,4-tetramethoxybenzene, 1,2,3,5-tetramethoxybenzene, 1,2,4,5-tetramethoxybenzene, 1-methoxy-2-methylbenzene, 1-methoxy-3-methylbenzene, 1-methoxy-4-methylbenzene, ethoxybenzene, and butoxybenzene.

The aromatic compound with one or more alkoxy functional groups forms an unstable thermodynamic phase with the compound with two or more alcohol functional groups at temperatures below 100° C., but it has high solubility for the polyol produced from depolymerization. Therefore, the resulting polyol is concentrated in the organic phase layer at a high concentration.

The organic phase layer, which contains most of the polyol produced from the depolymerization reaction, forms a clear phase separation over a wide temperature range with the hydrophilic layer, which is mainly composed of the compound with two or more alcohol functional groups. This allows for very simple, stable, and effective separation, and from this, a high-yield polyol product may be purified and obtained.

In addition, the aromatic compound with one or more alkoxy functional groups is a compound that can have a strong intermolecular interaction (π-π and hydrogen bonding) with the urethane bond and the adjacent polymer structure. When it is added to the depolymerization composition for urethane-functionalized polymers, it not only dissolves the urethane-functionalized polymer quickly but also promotes the nucleophilic attack of the catalyst and the compound with two or more alcohol functional groups (diol or polyol) on the urethane-functionalized polymer. As a result, the depolymerization reaction rate is enhanced, and the depolymerization is directed to have excellent selectivity for the polyol product. During the process of obtaining the converted monomer, the solvent and unreacted materials used in the reaction can be easily recovered and reused by simple physical separation methods such as filtration, distillation, evaporation, and extraction.

Therefore, when the depolymerization of a urethane-functionalized polymer is carried out using the depolymerization composition according to the present disclosure, the urethane-functionalized polymer dissolves quickly even without the addition of a depolymerization catalyst. It also allows for a relatively fast depolymerization rate at a low temperature (below 160° C.) where the rate of side reactions is slow, thereby enabling very effective control of the depolymerization reaction.

One of the features of the present disclosure is that the mixed solvent for the reaction to perform depolymerization comprises both one or more selected from the aromatic compound with one or more alkoxy functional groups and one or more selected from the compound with two or more alcohol functional groups.

Some or all of the compound with two or more alcohol functional groups may participate as a reactant in the depolymerization. As the urethane bond decomposes, it is added to the product to create a polyol product. The compound with two or more alcohol functional groups simultaneously plays the role of a reactant for depolymerization and a reaction medium that dissolves the urethane-functionalized polymer to allow the liquid-phase reaction to proceed. The aromatic compound with one or more alkoxy functional groups does not directly participate in the depolymerization reaction as a reactant. Instead, it plays the role of a cosolvent that increases the rate of liquefaction of the urethane-functionalized polymer by dissolving it and facilitates the nucleophilic attack of the compound with two or more alcohol functional groups by forming a strong interaction with the urethane bond as the depolymerization proceeds. As a result, the depolymerization reaction rate is enhanced at a low temperature.

In the mixed solvent, the weight ratio of the aromatic compound with one or more alkoxy functional groups to the compound with two or more alcohol functional groups is 1:20 to 20:1, preferably 1:10 to 10:1, and more preferably 1:2 to 2:1.

The depolymerization composition of the present disclosure may also further comprise a depolymerization catalyst for the urethane-functionalized polymer.

The catalyst may be anything that may enhance the depolymerization reaction rate of the urethane-functionalized polymer. It may be one or more selected from the group consisting of metal catalysts such as alkali hydroxides, alkaline-earth hydroxides, alkali acetates, alkaline-earth acetates, alkali carbonates, alkali bicarbonates, alkaline-earth carbonates, and alkali oxides, or guanidine-based or amine-based organic compounds, for decomposing the urethane bond.

The total mass of the catalyst in the depolymerization composition may be in the range of 0.0001 times to 0.5 times, and preferably in the range of 0.001 times to 0.1 times, the mass of the urethane-functionalized polymer when the depolymerization composition is applied to the depolymerization of the urethane-functionalized polymer.

The present disclosure also provides a method for depolymerizing a urethane-functionalized polymer.

The depolymerization method according to the present disclosure may be characterized by contacting a mixed solvent of a compound with two or more alcohol functional groups and an aromatic compound with one or more alkoxy functional groups with the urethane-functionalized polymer to decompose the urethane bonds within the polymer. The types and mixing ratios of the compound with two or more functional groups and the aromatic compound with one or more alkoxy functional groups are the same as those described in the section on the depolymerization composition above, so the description will be omitted to avoid repetition.

In the method for depolymerizing the urethane-functionalized polymer according to the present disclosure, the mass of the initial polymer raw material may be adjusted to a ratio of 1 wt % to 200 wt % based on the mass of the mixed solvent for depolymerization, i.e., the mixed solvent containing the aromatic compound with one or more alkoxy functional groups and the compound with a polyhydric alcohol functional group. When the polymer raw material is introduced within this numerical range, a uniform reactant for depolymerization is formed, the reaction is maintained stably to ensure uniform product quality, and the productivity and economic viability of the urethane-functionalized polymer depolymerization process may be enhanced.

In the method for depolymerizing the urethane-functionalized polymer, the mixed solvent may be heated to a temperature of 100° C. or higher to allow the dissolution of the urethane-functionalized polymer. The reaction temperature for depolymerization may be the same as or higher than this temperature and may be carried out in the temperature range of 100° C. to 170° C., and preferably in the temperature range of 140° C. to 165° C.

In the present disclosure, the aforementioned catalyst may be added to enhance the depolymerization reaction rate of the urethane-functionalized polymer. The total mass of the catalyst added to the depolymerization may be in the range of 0.0001 times to 0.5 times, and preferably in the range of 0.001 times to 0.1 times, the mass of the urethane-functionalized polymer.

The catalyst for depolymerization may be added before or after heating the reaction mixture for depolymerization, and it may be added directly to the reaction mixture or dissolved in a portion of the solvent before being added.

In the method for depolymerizing a urethane-functionalized polymer and manufacturing polyol according to the present disclosure, since the depolymerization proceeds below the boiling point of the applied reaction solvent, a positive pressure may not be generated. However, if a low-boiling point solvent is used, depolymerization may be carried out under a low absolute pressure of 1.0 atm to 1.5 atm.

In the method for depolymerizing a urethane-functionalized polymer according to the present disclosure, the aromatic compound with an alkoxy functional group is used as a cosolvent. This has the characteristic of being able to significantly suppress the oxidation of the depolymerization product by side reactions. However, it is advantageous for the gas phase occupying the volume other than the reactants to be free of oxygen. Therefore, a step of purging with an inert gas such as helium, argon, or nitrogen through a repeated purging process may be carried out before or at the beginning of the reaction, or depolymerization may be performed under conditions where there is some gas flow.

In the method for depolymerizing a urethane-functionalized polymer according to the present disclosure, the depolymerization reaction time may vary depending on the amount and form of the applied polymer. However, when the aforementioned composition and conditions for the reactant are applied to perform rapid depolymerization, the polymer is predominantly decomposed at a very fast depolymerization reaction rate within 1 hour of the initial reaction time, and most of the initial urethane bonds are decomposed after a reaction time of 2 hours.

In controlling the depolymerization reaction time according to the present disclosure, the depolymerization may be allowed to proceed for 1 to 8 hours after the start of the reaction to ensure sufficient polymer decomposition. However, to secure a sufficient yield, quality, and productivity of the manufactured polyol, it is preferable to perform depolymerization for 2 to 4 hours.

In one embodiment according to the present disclosure, the aromatic compound with one or more alkoxy functional groups used has a very limited solubility with the compound with two or more alcohol functional groups used as a reactant at temperatures below 100° C., but it mixes well with the polyol produced from depolymerization. However, at the depolymerization reaction temperature range of 100° C. to 170° C., an thermodynamically discontinuous emulsion phase is formed or a single phase of reactant exists because the stirring of the reactant is accompanied. In particular, when most of the urethane bonds have been decomposed through depolymerization in the temperature range of 140° C. to 165° C., it is characterized by existing as a single phase of reactant.

In the method for depolymerizing a urethane-functionalized polymer according to the present disclosure, unreacted materials and solid foreign substances may be removed from the reaction product after the depolymerization reaction by various physical methods such as precipitation, filtration, flocculation, flotation, and pressing, either before or after the reaction is terminated, and can be reintroduced into the depolymerization. In the case of using filtration for removal, a filter with fine pores smaller than the particle size of the unreacted polymer may be used, and pressurization or depressurization may be performed to increase the flow rate of the filtrate.

When the liquid reaction mixture obtained as a result of depolymerization is taken and its temperature is lowered again to below 100° C., phase separation occurs. Most of the unreacted compound with two or more alcohol functional groups is concentrated in the hydrophilic solution layer and may be easily recovered by a simple physical separation process. The liquid mixed product containing the polyol, which is separated into a different phase from the hydrophilic solution layer, may be obtained as a high-purity polyol as a final product by removing the cosolvent using relatively simple separation methods such as evaporation, distillation, and extraction.

To induce a clearer phase separation, one or more polar and non-polar solvents may be additionally added to the reaction mixture obtained from the depolymerization.

The recycled polyol obtained from the separation process may be used as a part or all of the raw material for re-polymerization and may be used in the synthesis of new polyurethane materials.

Some or all of the solvent recovered from the separation process may be reused as a part of the composition required for the mixed solvent for the preceding depolymerization reaction.

The present disclosure also provides a method for manufacturing polyol by depolymerizing a urethane-functionalized polymer.

The method for manufacturing polyol is characterized by comprising a step of contacting a reaction mixture of a compound with two or more alcohol functional groups and an aromatic compound with one or more alkoxy functional groups with the urethane-functionalized polymer to decompose the urethane bonds within the polymer and obtain polyol.

The composition and conditions related to the method are the same as those for the method of depolymerizing a urethane-functionalized polymer, so the description will be omitted.

Hereinafter, the details of the present disclosure will be explained through examples, comparative examples, and experimental examples. These are representative examples related to the present disclosure, and it is made clear that they do not limit the scope of the present disclosure.

### Raw Material 1 (Waste Polyurethane)

Flexible waste polyurethane foam recovered from a used mattress was cut and used. After washing with an excess of ethanol and water and drying completely, the polyurethane was cut and pulverized into chips with a cross-sectional length of 2 mm or less, and prepared as Raw Material 1.

### Raw Material 2 (Virgin Polyol Raw Material)

The polyether polyol (OH value=56 mgKOH/g, viscosity=480 cP) raw material used to manufacture Raw Material 1 was supplied by the manufacturer and prepared as Raw Material 2 without separate purification.

### Raw Material 3 (Polyol Raw Material for Polyurethane Synthesis)

Raw Material 3 was prepared as a polyol for recycled urethane polymerization by adding 0.2 wt % of water, 2 wt % of a urethane polymerization catalyst, 1.5 wt % of a surfactant, and 2 wt % of a foaming agent to Raw Material 2's polyether polyol, all based on 100 wt % of the total composition.

### Raw Material 4 (Diisocyanate Raw Material)

The liquid-modified polymeric methylene diphenyl diisocyanate (MDI) raw material (NCO value=33%, viscosity=17 cP) used to manufacture Raw Material 1 was supplied by the manufacturer and prepared as Raw Material 4 without separate purification.

### Comparative Example 1

Raw Material 2 polyol was weighed at a mass ratio of 1:4 with Raw Material 3, which was a blend of additives, and mixed for about 2 hours using a magnetic stirrer to prepare a mixed polyol raw material for polyurethane synthesis. A disperser tool (S25N-18G) connected to a homogenizer (IKA ULTRA-TURRAX) was adjusted to a rotor speed of 28,000 rpm. The solution was mixed with 12 g of the previously prepared mixed polyol raw material and 3 g of isocyanate from Raw Material 4. The solution was then quickly transferred to a 50 ml polypropylene container with an inner diameter of 33 mm and was observed for the formation of polyurethane foam. After 24 hours of exposure to the atmosphere, the manufactured foam was cut along the longitudinal axis of the growth direction to ensure that the cut surfaces were symmetrical and each cut foam had the same shape. The cut surface was observed by enlarging it with a stereo microscope, and the shape of the foam layer formed during the polyurethane foam manufacturing process was confirmed.

### Comparative Example 2

### (a) Depolymerization of Polyurethane (Urethane-Functionalized Polymer)

About 20.0 g of polyurethane foam from Raw Material 1 was used as the polymer raw material for depolymerization. The weighed polyurethane foam was added to a 500 mL 3-neck flask, and 18 g of anhydrous ethylene glycol was added without a cosolvent to prepare the initial reactant for depolymerization. A condenser with an internal thermocouple for temperature measurement and a septum made of Teflon-silicone to sample liquid were connected to the side necks of the 3-neck flask via bushing-type adapters. A Teflon seal connecting a stirring rod rotated by an external overhead stirrer to an internal impeller was connected to the central neck of the 3-neck flask to prepare a depolymerization reactor isolated from the outside atmosphere. This was placed in a thermostatic bath filled with high-temperature methyl phenyl silicone oil, which was maintained at a constant temperature by a PID temperature controller. The mixture was stirred at a speed of 250 rpm until the temperature inside the reactor reached 200° C. When the temperature inside the reactor was stabilized, a catalyst solution, prepared by adding 0.1 g of sodium hydroxide (NaOH) to 2 g of the prepared ethylene glycol, was added to start the reaction. The FT-IR spectra (ATR/FT-IR; Bruker ALPHA II) of polyurethane, the initial polyol used for urethane synthesis, and the recycled polyol obtained by taking a portion of the reactant during the depolymerization process were measured and observed to determine whether the depolymerization was complete.

### (b) Recovery of Reaction Solvent

After reacting for 4 hours at 200° C., the reactor was separated from the oil thermostatic bath to terminate the reaction. When the temperature dropped to below 100° C. and the boundaries of the phases were clearly observed, the unreacted ethylene glycol and a number of catalysts in the lower phase were recovered using a separatory funnel. The organic compound separated in the upper phase was transferred to a 100 ml evaporating flask with a known tare weight and measured.

### (c) Obtaining Polyol Product

The 100 ml evaporating flask containing the separated upper phase mixture was attached to a rotary evaporator and was continuously contacted with a thermostatic water bath maintained at 65° C. The flask was rotated at a speed of 150 rpm under reduced pressure (10 torr) for about 1 hour to completely remove the distillate. About 13.18 g of a highly viscous, opaque, black solution remaining in the rotary evaporator was obtained as the polyol product.

### (d) Analysis of Polyol Product Properties

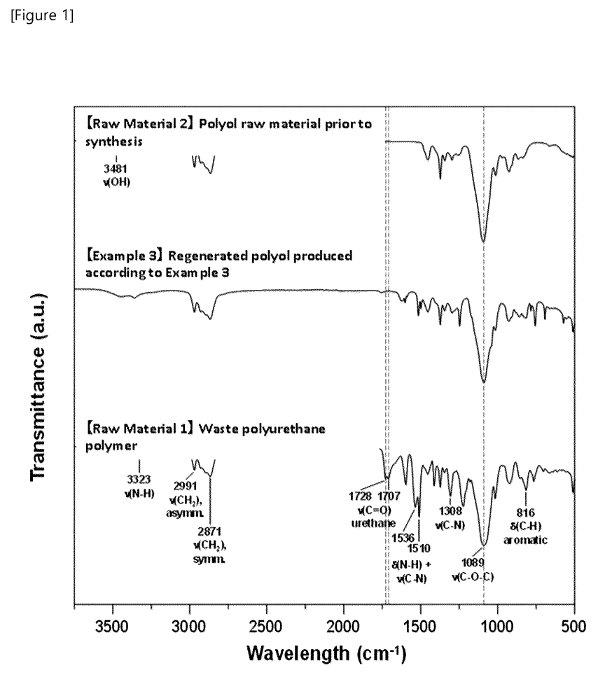

To analyze the bonding structure of the functional groups of the final recycled polyol (the product obtained from the depolymerization of Raw Material 1) according to the depolymerization process in (a), a portion was taken as an ATR sample and FT-IR analysis was performed. The result is shown in FIG. 1.

Meanwhile, the structural properties of the initial polyol used for polyurethane synthesis (Raw Material 2) and the recycled polyol were compared using 1H-NMR. The 1H-NMR sample was prepared by diluting the polyol with N,N-Dimethylformamide (DMF)-d7 as a solvent to a final concentration of 6.25%. The viscosity of the initial polyol (Raw Material 2) and the recycled polyol (the product obtained from the depolymerization of Raw Material 1) was measured using an automatic rotary viscometer (IKA ROTAVISC lo-vi with VOLS-1). The acid value and OH value (hydroxyl value) of the manufactured polyol were measured by taking a portion of the manufactured recycled polyol product and following the procedures and methods specified in ASTM D4662-20 and ASTM D4274-21. In addition, the color characteristics of the manufactured recycled polyol were measured using a spectrophotometer (manufacturer: Konica Minolta, model: CM-3600A).

### (e) Synthesis of Recycled Polyurethane

A polyurethane foam was manufactured and analyzed by the same method and procedure as in Comparative Example 1, except that the recycled polyol manufactured according to the depolymerization process in (a) was applied as the raw material for polyurethane synthesis instead of the polyol of Raw Material 2.

### Comparative Example 3

The depolymerization reaction was carried out by the same method and procedure as in Comparative Example 2, except that a step was added in the ‘(a) Depolymerization of Polyurethane’ stage of Comparative Example 2. Before isolating from the outside atmosphere after preparing the initial reactants for depolymerization, nitrogen was flowed at a rate of 100 sccm for 45 minutes to remove oxygen from the inside of the reactor. Approximately 13.20 g of an opaque, dark brown polyol was finally obtained through the same purification method as in Comparative Example 2 (c). The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 1

The depolymerization reaction was carried out by the same method and procedure as in Comparative Example 2, except that in the ‘(a) Depolymerization of Polyurethane’ stage of Comparative Example 2, 18 g of anhydrous ethylene glycol and 14 g of anisole were weighed and prepared as the initial reactant for depolymerization for about 20.0 g of polyurethane foam from Raw Material 1, the amount of sodium hydroxide (NaOH) was doubled (0.2 g), and the depolymerization was exposed to a low-temperature (160° C.) reaction condition. The same method as in Comparative Example 2 (c) was applied, but the cosolvent was also removed to finally obtain approximately 14.69 g of a translucent polyol with a pale yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 2

The depolymerization reaction was carried out by the same method and procedure as in Comparative Example 3, except that in the depolymerization stage of Comparative Example 3, 18 g of anhydrous ethylene glycol and 14 g of anisole were weighed and prepared as the initial reactant for depolymerization for about 20.0 g of polyurethane foam from Raw Material 1, a step of flowing nitrogen at a rate of 100 sccm for 45 minutes before isolating from the outside atmosphere (as in Comparative Example 3) was added to remove oxygen from the inside of the reactor, and the depolymerization was exposed to a low-temperature (160° C.) reaction condition. The same method as in Comparative Example 2 (c) was applied, but the cosolvent was also removed to finally obtain approximately 14.55 g of a transparent polyol with a pale yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 3

The depolymerization reaction was carried out by the same method and procedure as in Example 2, except that a catalyst solution was used, which was prepared by dissolving 0.1 g of potassium hydroxide (KOH) instead of sodium hydroxide (NaOH) in 2 g of ethylene glycol. The same method as in Comparative Example 2 (c) was applied, but the cosolvent was also removed to finally obtain approximately 15.39 g of a transparent polyol with a pale yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 4

The depolymerization reaction was carried out by the same method and procedure as in Example 2, except that a catalyst solution was used, which was prepared by dissolving 0.01 g of triazabicyclodecene (TBD) instead of sodium hydroxide (NaOH) in 2 g of ethylene glycol. The same method as in Comparative Example 2 (c) was applied, but the cosolvent was also removed to finally obtain approximately 14.96 g of a transparent polyol with a pale yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 5

The depolymerization reaction was carried out by the same method and procedure as in Example 2, except that a catalyst solution was used, which was prepared by dissolving 0.1 g of potassium carbonate (K₂CO₃) instead of sodium hydroxide (NaOH) in 2 g of ethylene glycol. The same method as in Comparative Example 2 (c) was applied, but the cosolvent was also removed to finally obtain approximately 14.50 g of a transparent polyol with a pale yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 6

The depolymerization reaction was carried out by the same method and procedure as in Example 2, except that a catalyst solution was used, which was prepared by dissolving 0.1 g of potassium bicarbonate (KHCO₃) instead of sodium hydroxide (NaOH) in 2 g of ethylene glycol. The same method as in Comparative Example 2 (c) was applied, but the cosolvent was also removed to finally obtain approximately 15.54 g of a transparent polyol with an orange color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 7

The depolymerization reaction was carried out by the same method and procedure as in Example 2, except that a catalyst solution was used, which was prepared by dissolving 0.1 g of potassium acetate (CH₃COOK) instead of sodium hydroxide (NaOH) in 2 g of ethylene glycol. The same method as in Comparative Example 2 (c) was applied, but the cosolvent was also removed to finally obtain approximately 7.64 g of a transparent polyol with a slightly dark yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 8

The depolymerization reaction was carried out by the same method and procedure as in Example 2, except that a catalyst solution was used, which was prepared by dissolving 0.2 g of potassium acetate (CH₃COOK) instead of sodium hydroxide (NaOH) in 2 g of ethylene glycol. The same method as in Comparative Example 2 (c) was applied, but the cosolvent was also removed to finally obtain approximately 14.94 g of a transparent polyol with a pale orange color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 9

The depolymerization reaction was carried out by the same method and procedure as in Example 2, except that in the ‘(a) Depolymerization of Polyurethane’ stage, 18 g of diethylene glycol and 14 g of anisole were weighed and prepared as the initial reactant for depolymerization instead of ethylene glycol for about 20.0 g of polyurethane foam from Raw Material 1, and a catalyst solution was prepared by adding 0.1 g of potassium hydroxide (KOH) to 2 g of diethylene glycol instead of ethylene glycol to start the reaction. The same method as in Comparative Example 2 (c) was applied, but the cosolvent was also removed to finally obtain approximately 15.22 g of a transparent polyol with a dark brown color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 10

The depolymerization reaction was carried out by the same method and procedure as in Example 2, except that in the ‘(a) Depolymerization of Polyurethane’ stage, 18 g of glycerol and 14 g of anisole were weighed and prepared as the initial reactant for depolymerization instead of ethylene glycol for about 20.0 g of polyurethane foam from Raw Material 1, and a catalyst solution was prepared by adding 0.1 g of potassium hydroxide (KOH) to 2 g of glycerol instead of ethylene glycol to start the reaction. The same method as in Comparative Example 2 (c) was applied, but the cosolvent was also removed to finally obtain approximately 15.82 g of an opaque polyol with a pale yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 11

The depolymerization reaction was carried out by the same method and procedure as in Example 2, except that in the ‘(a) Depolymerization of Polyurethane’ stage, 14 g of ethoxybenzene was weighed and added as a cosolvent instead of anisole for about 20.0 g of polyurethane foam from Raw Material 1, and a catalyst solution was prepared by adding 0.1 g of potassium carbonate (K₂CO₃) instead of sodium hydroxide (NaOH) to 2 g of ethylene glycol to start the reaction.

In the polyol purification process, the cosolvent was removed by a solvent extraction method to obtain the polyol. 50 ml of n-hexane was added to the reactant and stirred at room temperature for 24 hours to remove the cosolvent dissolved in n-hexane. Then, 50 ml of toluene was added to induce phase separation of the depolymerization reactant. The organic phase, which was mainly composed of toluene and polyol, was transferred to a 100 ml evaporating flask with a known tare weight and measured. The same method as in Comparative Example 2 (c) was applied, but toluene was also removed through the evaporation process to finally obtain approximately 13.80 g of an opaque polyol with a dark yellow color. The properties of the manufactured polyol were analyzed by the same method and procedure as in Comparative Example 2 (d). In addition, a polyurethane foam was manufactured and its properties were observed by the same method and procedure as in Comparative Example 2 (e), except that the recycled polyol manufactured according to the depolymerization process was applied as the raw material for polyurethane synthesis instead of the polyol of Raw Material 2.

### Example 12

The depolymerization reaction was carried out by the same method and procedure as in Example 11, except that in the ‘(a) Depolymerization of Polyurethane’ stage, 14 g of 1,4-dimethoxybenzene was weighed and added as a cosolvent instead of ethoxybenzene for about 20.0 g of polyurethane foam from Raw Material 1. The same method as in Comparative Example 2 (c) was applied, but the solvent was completely removed through the evaporation process to finally obtain approximately 14.15 g of an opaque polyol with a dark yellow color. A recycled polyol product was obtained by the same method and procedure as in Example 11, and the properties of the product were analyzed.

TABLE 1

| | | | | Initial | | | | | | |
|---|---|---|---|---|---|---|---|---|---|---|
| | Catalyst/ | | | Nitrogen | Mass of | | | | OH | Acid |
| | Amount Used | Reaction | | Purge | Recovered | Yellowness Index | | Viscosity | value | value |
| | (per unit mass | Temperature | Reaction | (Presence/ | Polyol | ASTM | ASTM | (25° C., | (mg | (mg |
| Category | of waste PU) | (° C.) | Solvent* | Absence) | (g) | E313-73 | D1925 | cP) | KOH/g) | KOH/g) |
| Raw | — | — | — | — | — | 0.0 | 0.4 | 480 | 59 | 0.03 |
| Material 2 | | | | | | | | | | |
| Comparative | NaOH/ | 200 | EG | X | 13.18 | 99.2 | 283 | 1968 | 310 | 0.11 |
| Example 2 | 0.005 | | | | | | | | | |
| Comparative | NaOH/ | 200 | EG | ◯ | 13.20 | 99.3 | 280 | 1971 | 313 | 0.15 |
| Example 3 | 0.005 | | | | | | | | | |
| Example 1 | NaOH/ | 160 | EG | X | 14.69 | 92.3 | 129 | 711 | 199 | 0.10 |
| | 0.010 | | | | | | | | | |
| Example 2 | NaOH/ | 160 | EG | ◯ | 14.55 | 87.6 | 111 | 1275 | 280 | 0.09 |
| | 0.005 | | | | | | | | | |
| Example 3 | KOH/ | 160 | EG | ◯ | 15.39 | 84.8 | 107 | 696 | 213 | 0.09 |
| | 0.005 | | | | | | | | | |
| Example 4 | TBD/ | 160 | EG | ◯ | 14.96 | 81.9 | 103 | 709 | 204 | 0.05 |
| | 0.005 | | | | | | | | | |
| Example 5 | K₂CO₃/ | 160 | EG | ◯ | 14.50 | 78.2 | 94 | 727 | 226 | 0.02 |
| | 0.005 | | | | | | | | | |
| Example 6 | KHCO₃/ | 160 | EG | ◯ | 15.54 | 85.5 | 108 | 701 | 228 | 0.05 |
| | 0.005 | | | | | | | | | |
| Example 7 | KOAc/ | 160 | EG | ◯ | 7.14 | 84.3 | 106 | 762 | 255 | 0.08 |
| | 0.005 | | | | | | | | | |
| Example 8 | KOAc/ | 160 | EG | ◯ | 14.94 | 80.2 | 00 | 717 | 231 | 0.05 |
| | 0.010 | | | | | | | | | |
| Example 9 | KOH/ | 160 | DEG | ◯ | 15.22 | 96.2 | 146 | 514 | 191 | 0.09 |
| | 0.005 | | | | | | | | | |
| Example 10 | KOH/ | 160 | Gly | ◯ | 15.82 | 84.2 | 104 | 1090 | 139 | 0.14 |
| | 0.005 | | | | | | | | | |

*EG: ethylene glycol, DEG: Diethylene glycol, Gly: Glycerol

Table 1 compares the reaction conditions for manufacturing recycled polyol according to the depolymerization method of the present disclosure and the conventional high-temperature depolymerization method, as well as the yield and properties of the polyol manufactured therefrom. The dominant depolymerization reaction was observed to proceed within 2 hours of reaction time, and most of the polyurethane was decomposed. However, the reactants were exposed to the depolymerization conditions for 4 hours to induce a sufficient depolymerization reaction and then purified to obtain a recycled polyol product.

Information on the structural changes of the polymer compound following the depolymerization of polyurethane was obtained by comparing the final spectrum of the obtained recycled polyol with the spectra obtained for polyurethane and the initial polyol raw material and observing the changes in the characteristic peaks.

FIG. 1 illustrates the ATR/FT-IR spectra of waste polyurethane (Raw Material 1), the initial polyol (Raw Material 2) used to synthesize it, and the recycled polyol obtained from the depolymerization of Example 3.

In the spectrum for polyurethane (Raw Material 1), the absorption peaks observed at wavelengths of 1707 cm⁻¹ and 1728 cm⁻¹ correspond to the characteristic peaks of the carbonyl (C═O) group in the urethane bond. In the intermediate product where partial decomposition occurred during the depolymerization process, their attenuation was observed, and in the spectrum for the recycled polyol obtained from complete depolymerization, the characteristic peaks representing the urethane bond were not observed. The end of the depolymerization of waste polyurethane could be determined from the changes in these characteristic peaks.

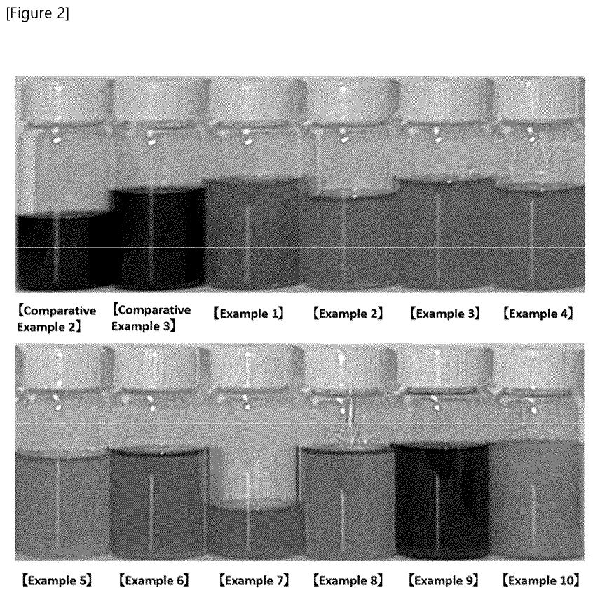

The recycled polyol samples shown in Table 1 are each compared in the photograph of FIG. 2. In the case of the polyol obtained from high-temperature depolymerization without a cosolvent (Comparative Examples 2 and 3), it had a dark brown or black color. In contrast, in the case where waste polyurethane was decomposed through cosolvent-based low-temperature depolymerization according to the examples of the present disclosure, a transparent and light-colored recycled polyol was obtained as the final product of the depolymerization reaction and purification process in most cases. Although not shown in Table 1, when the same depolymerization reaction conditions were applied, including the temperature (160° C. or lower), but without a cosolvent or using an aromatic compound without an alkoxy functional group, such as toluene or p-xylene, the depolymerization of polyurethane hardly proceeded.

When depolymerization is carried out with ethylene glycol as a reaction solvent in the presence of a catalyst at a high temperature, and a high amount of thermal energy is supplied, depolymerization proceeds at a relatively high rate. However, side reactions such as thermal degradation and hydrolysis by oxygen may also proceed rapidly at the same time during the depolymerization process. These side reactions are known to cause discoloration by generating chromophore and colorant functional groups. In particular, in the case of a polymer with urethane bonds formed from an isocyanate with an aromatic structure, such as TDI or MDI, discoloration is known to be exacerbated when side reactions caused by thermal degradation or hydrolysis occur, as the functional groups causing the discoloration have a greater effect on the electronic structure of the aromatic ring.

However, when an alkoxy-bonded aromatic compound is used as a cosolvent according to the present disclosure, it is expected that the strong intermolecular interactions such as hydrogen bonding and π-π bonding will not only significantly lower the reaction activation energy for the decomposition of the urethane bond but also greatly reduce the diffusion rate of oxygen or moisture that promotes discoloration during the decomposition process, as the hydrophobic cosolvent is present at a high density near the urethane bond. From this, the transesterification reaction of the glycol may proceed more selectively at the same time as the urethane functional group is decomposed, and a high-quality depolymerization product can be obtained.

The depolymerization process of polyurethane is a heating reaction process for decomposition at a high temperature, so it may be advantageous to remove oxygen in the initial stage to control the degree of discoloration. A catalyst that may enhance the rate of the transesterification reaction may also be advantageous.

According to the results of Comparative Examples 2 and 3, it may be seen that it is not easy to control the side reactions that cause discoloration in depolymerization reactions performed at very high temperatures. In contrast, in the case of the recycled polyols manufactured from Examples 1 and 2, where depolymerization was carried out using a cosolvent, there was some difference in the yield and quality of the final polyol obtained when the amount of catalyst was increased or when the reaction was carried out in an initial vacuum atmosphere to control the rate and selectivity of the transesterification reaction.

When the amount of catalyst was increased (Example 1), the waste polyurethane was almost completely decomposed, showing a very high recovery rate close to the mass of the polyol used for initial polymer synthesis (about 75% of the polyurethane, 15 g), and it showed a low viscosity. However, since the depolymerization was carried out without removing oxygen in the initial stage of the reaction, a higher degree of yellowing occurred in the final polyol product.

Examples 2 to 7 compare the results of depolymerization carried out by using alkali metal hydroxides, alkali metal carbonates, alkali metal bicarbonates, alkali metal acetates, or guanidine-based organic compounds as catalysts while maintaining the same mass of catalyst added per unit mass of the waste polyurethane used as the depolymerization raw material.

Except for the case where potassium acetate was used as a catalyst, polyol was obtained at a very high yield. Unlike in Comparative Examples 2 and 3, the color of the obtained polyol was all transparent yellow, and the OH Value (mg KOH/g) was in the range of 200 to 300, and it showed a low viscosity.

Meanwhile, in the case of using potassium acetate as a catalyst, it was found that a similar depolymerization performance and a high-quality recycled polyol were obtained in the depolymerization reaction carried out by increasing the amount of catalyst (Example 8).

In the case where polyhydric alcohols such as diethylene glycol or glycerol instead of ethylene glycol were used as the reaction solvent and applied to the cosolvent-based low-temperature depolymerization according to the present disclosure (Examples 9 and 10), most of the polyol obtainable from waste polyurethane could be recovered. However, a different type of recycled polyol product was obtained that showed some differences in physical properties (color, viscosity, etc.) from the initial polyol.

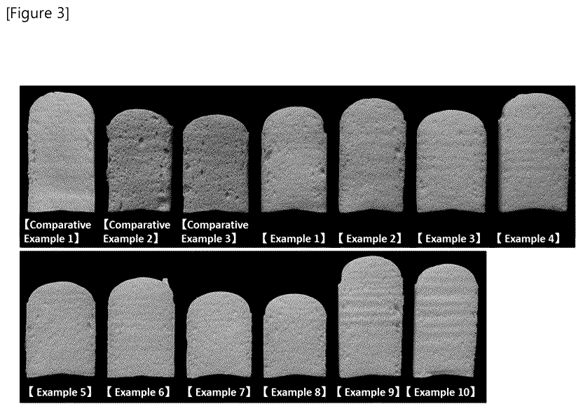

FIG. 3 shows the cross-section of polyurethane foam synthesized using different polyol raw materials. Comparative Example 1 used initial (virgin) polyol raw material (Raw Material 2), Comparative Examples 2 and 3 used recycled polyol manufactured from high-temperature depolymerization, and Examples 1 to 10 used recycled polyol obtained from cosolvent-based depolymerization according to the present disclosure.

Comparative Examples 2 and 3, which used recycled polyol obtained from high-temperature depolymerization, showed low reactivity and had a very rough and diverse-shaped foam layer during the foaming process. In contrast, they were confirmed to be dark in color compared to the polyurethane foam synthesized from the recycled polyol manufactured from the examples.

In contrast, the polyurethane foam synthesized using the recycled polyol manufactured according to the present disclosure had a relatively uniform foam layer formation. In terms of reactivity and foam color, many of them were indistinguishable from those manufactured with initial (virgin) polyol raw material.

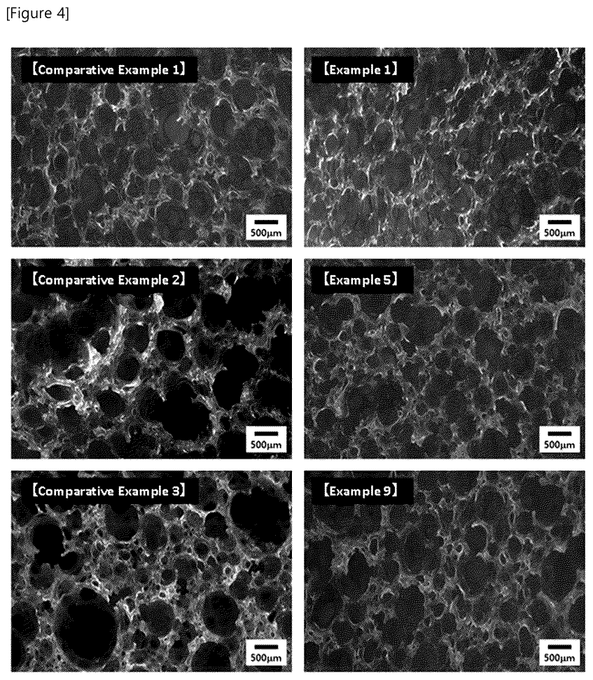

FIG. 4 illustrates a local view of the foam layer formed inside some polyurethane foam samples, observed with a stereo microscope. The polyurethane synthesized using the recycled polyol manufactured according to the present disclosure (Examples 1, 5, and 9) formed a relatively uniform foam layer, and a shape similar to that using initial (virgin) polyol raw material (Comparative Example 1) was observed. In contrast, a uniform foam layer was not formed in the polyurethane foam obtained from high-temperature depolymerization without a cosolvent (Comparative Examples 2 and 3).

TABLE 2

| | Cosolvent | | | | | | |
|---|---|---|---|---|---|---|---|
| | Type and | | | | | OH | Acid |
| | Amount Used | Mass of | Yellowness Index | | Viscosity | value | value |
| | (per unit mass | Recovered | ASTM | ASTM | (25° C., | (mg | (mg |
| Category | of waste PU) | Polyol (g) | E313-73 | D1925 | cP) | KOH/g) | KOH/g) |
| Raw | — | — | 0.0 | 0.4 | 480 | 59 | 0.03 |
| Material 2 | | | | | | | |
| Example 5 | Anisole/ | 14.50 | 78.2 | 94 | 727 | 226 | 0.02 |
| | 0.005 | | | | | | |
| Example 11 | EB*/ | 15.82 | 93.6 | 135 | 1823 | 170 | 0.18 |
| | 0.005 | | | | | | |
| Example 12 | 1,4-DMB**/ | 15.22 | 94.1 | 138 | 2013 | 297 | 0.09 |
| | 0.005 | | | | | | |

[Depolymerization Conditions] Catalyst and Amount Used (per 1 g of waste PU): K₂CO₃ 0.005 g, Reaction Temperature: 160° C., Reaction Solvent: EG 1 g
*EB: ethoxy benzene,
**DMB: dimethoxy benzene

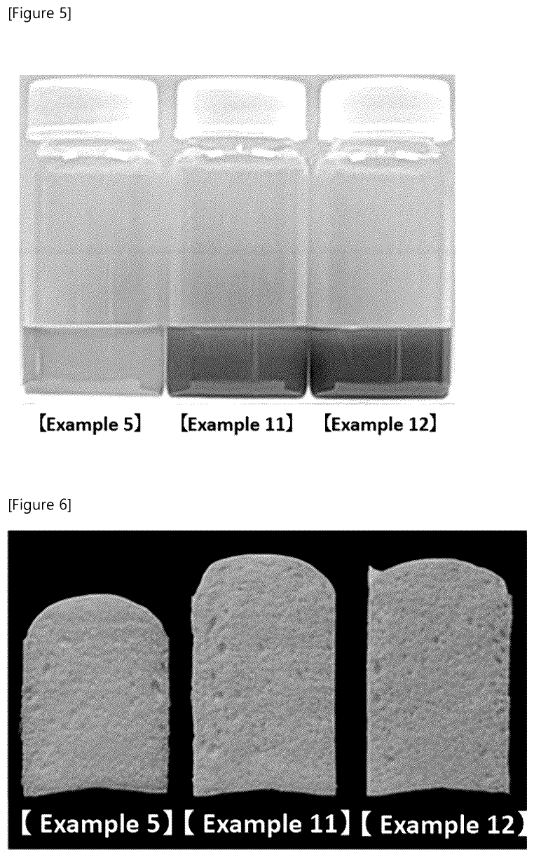

Table 2 illustrates the yield and properties of the polyol obtained by carrying out the depolymerization with different types of cosolvents. Photographs of the obtained recycled polyol samples are each compared in FIG. 5.

In Examples 11 and 12, an aromatic compound with a higher number of carbon atoms in the alkoxy functional group or with multiple alkoxy functional groups bonded to the aromatic ring was applied as a cosolvent. Similar to the previously performed examples, complete decomposition of waste polyurethane occurred within 4 hours of reaction time through low-temperature depolymerization, and the manufactured recycled polyol was recovered in a mass close to the maximum yield (the polyol used for polyurethane synthesis was about 15 g).

Meanwhile, the polyol obtained from Examples 11 and 12 was a more highly viscous product with a darker color compared to the case of Example 5, which used anisole for depolymerization.

FIG. 6 compares the cross-section of the polyurethane foam synthesized using the polyol obtained from Examples 11 and 12 with the one obtained from Example 5. A somewhat rough foam layer was observed to be generated during the foaming process. However, a polyurethane foam with improved quality could be manufactured compared to the case using polyols obtained at high temperatures from Comparative Examples 2 and 3.

The specific parts of the content of the present disclosure have been described in detail above. It is clear that these specific descriptions are only preferred embodiments and do not limit the scope of the present disclosure to one of ordinary skill in the art. Therefore, the substantial scope of the present disclosure will be defined by the appended claims and their equivalents.

1. A depolymerization composition for polymer containing urethane-functional group, the composition comprises:
(1) a compound with two or more alcohol functional groups (—OH); and
(2) an aromatic compound with one or more alkoxy functional groups.

2. The depolymerization composition for the polymer containing urethane-functional group of claim 1, wherein the compound with two or more alcohol functional groups is one or more selected from the group consisting of ethylene glycol, trimethylene glycol, 1,2-propanediol, tetramethylene glycol, neopentyl glycol, pentamethylene glycol, hexamethylene glycol, decamethylene glycol, dodecamethylene glycol, 1,4-cyclohexanedimethanol, diethylene glycol, triethylene glycol, tetraethylene glycol, polyethylene glycol, dipropylene glycol, tripropylene glycol, tetrapropylene glycol, polypropylene glycol, di(tetramethylene) glycol, tri(tetramethylene) glycol, polytetramethylene glycol, pentaerythritol, 2,2-bis(4-β-hydroxyethoxyphenyl) propane, glycerol, pentanetriol, and hexanetriol.

3. The depolymerization composition for the polymer containing urethane-functional group of claim 1, wherein the aromatic compound with one or more alkoxy functional groups is one or more selected from the group consisting of methoxybenzene, 1,2-dimethoxybenzene, 1,3-dimethoxybenzene, 1,4-dimethoxybenzene, 1,2,3-trimethoxybenzene, 1,2,4-trimethoxybenzene, 1,3,5-trimethoxybenzene, 1,2,3,4-tetramethoxybenzene, 1,2,3,5-tetramethoxybenzene, 1,2,4,5-tetramethoxybenzene, 1-methoxy-2-methylbenzene, 1-methoxy-3-methylbenzene, 1-methoxy-4-methylbenzene, ethoxybenzene, and butoxybenzene.

4. The depolymerization composition for the polymer containing urethane-functional group of claim 1, wherein the compound with two or more alcohol functional groups and the aromatic compound with one or more alkoxy functional groups are mixed in a weight ratio of 1:20 to 20:1.

5. The depolymerization composition for the polymer containing urethane-functional group of claim 1, wherein the depolymerization composition further comprises one or more depolymerization catalysts for polymer containing urethane-functional group selected from the group consisting of metal catalysts such as alkali hydroxides, alkaline-earth hydroxides, alkali acetates, alkaline-earth acetates, alkali carbonates, alkali bicarbonates, alkaline-earth carbonates, and alkali oxides, or guanidine-based or amine-based organic compounds.

6. A method for depolymerizing a polymer containing a urethane-functional group, the method comprises:
contacting a mixed solvent of a compound having two or more alcohol-functional groups and an aromatic compound having one or more alkoxy-functional groups with the polymer containing a urethane-functional group.

7. The method for depolymerizing a polymer containing a urethane-functional group of claim 6, wherein the mass of the polymer containing the urethane-functional group is in the range of 1 wt % to 200 wt % based on the mass of the mixed solvent of the compound having two or more alcohol-functional groups and the aromatic compound having one or more alkoxy-functional groups.

8. The method for depolymerizing a polymer containing a urethane-functional group of claim 6, wherein the mixed solvent is in a temperature range of 100° C. to 170° C.

9. The method for depolymerizing a polymer containing a urethane-functional group of claim 6, wherein the contacting the mixed solvent of the compound having two or more alcohol-functional groups and the aromatic compound having one or more alkoxy-functional groups with the polymer containing the urethane-functional group is performed in the presence of one or more catalysts selected from the group consisting of metal catalysts such as alkali hydroxides, alkaline earth hydroxides, alkali acetates, alkaline earth acetates, alkali carbonates, alkali bicarbonates, alkaline earth carbonates, alkali oxides, or guanidine-based or amine-based organic compounds.

10. The method for depolymerizing a polymer containing a urethane-functional group of claim 9, wherein the total mass of the catalyst is in the range of 0.0001 times to 0.5 times relative to the mass of the polymer containing the urethane-functional group.

11. The method for depolymerizing a polymer containing a urethane-functional group of claim 6, wherein purging using an inert gas is performed before or at the initial stage of contacting the mixed solvent of the compound having two or more alcohol-functional groups and the aromatic compound having one or more alkoxy-functional groups with the polymer containing the urethane-functional group.

12. The method for depolymerizing a polymer containing a urethane-functional group according to claim 6, wherein phase separation is induced by cooling the generated liquid reaction mixture after the contacting.

13. A method for producing polyol by depolymerization of a polymer containing a urethane-functional group, the method comprises:
contacting a mixed solvent of a compound having two or more alcohol-functional groups and an aromatic compound having one or more alkoxy-functional groups with the polymer containing the urethane-functional group to decompose the urethane bond in the polymer and obtain polyol.
